(19) United States
(12) Patent Application Publication
(10) Pub. No.: US 2018/0303378 A1
(43) Pub. Date: Oct. 25, 2018

(54) Predictive Disease Breath Database Systems and Methods

(71) Applicant: Volatile Analysis Corporation, Grant, AL (US)

(72) Inventor: Katherine Bazemore, Grant, AL (US)

(21) Appl. No.: 15/961,787

(22) Filed: Apr. 24, 2018

Related U.S. Application Data
(60) Provisional application No. 62/489,062, filed on Apr. 24, 2017.

(51) Int. Cl. A61B 5/08; G01N 33/497; A61B 5/00; G06F 19/28

(52) U.S. Cl. CPC A61B 5/082; G01N 33/497; A61B 5/4842; G06F 19/28; A61B 5/7275

(57) ABSTRACT

A predictive disease breath database system (PDBDS) may accumulate information about the volatile, semi-volatile, and non-volatile organic compounds in breath/saliva. Such information may be analyzed over time to identify disease indications as early as possible, using non-invasive data collection via breath and alert patients directly for follow-up with a health professional.

### CROSS REFERENCE TO RELATED APPLICATION

This application claims priority to U.S. Provisional Application No. 62/489,062, entitled “Automated Disease Identification Platform” and filed on Apr. 24, 2017, which is incorporated herein by reference.

### RELATED ART

Various techniques for detecting disease have been developed and are instrumental in healthcare. Early detection is important and even sometimes critical in successful treatment for many types of diseases, but such early detection can be difficult. In addition, due to inherent difficulties in detecting many types of diseases, patients are sometimes given incorrect or inadequate diagnosis, which can lead to complications or problems in treatment. Moreover, improved techniques for detecting disease are generally desired.

### BRIEF DESCRIPTION OF THE DRAWINCIS

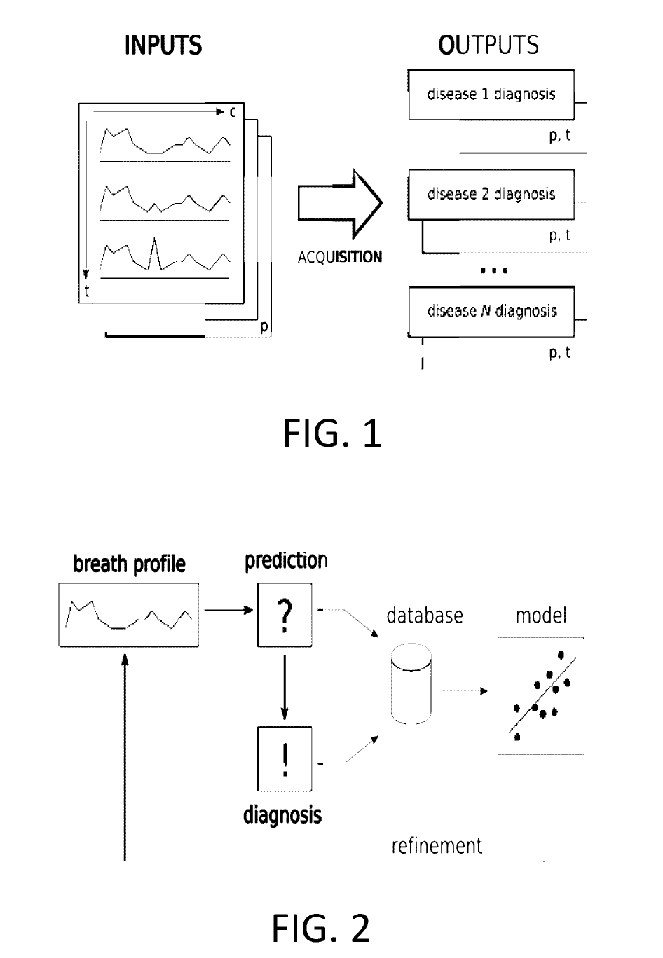

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views. FIG. 1 shows exemplary inputs and outputs of a predictive disease breath database system (PDBDS). The INPUT data is accumulated using company technology. Compounds [c] are plotted by concentration, at multiple time snapshots [t], for multiple patients [p]. The OUTPUT data is generated by doctors making a diagnosis of some number of disease conditions, each of which is associated with a patient [p] at time [t]. These data are suitable for the parameter/result format used for supervised machine learning techniques.

FIG. 2 shows an exemplary cycle for disease prediction. The prediction cycle involves collecting breath profiles on a regular basis, and escalating to actual medical diagnoses on a less frequent basis. The profiles and diagnoses (both positive and negative) are collected in the database, and are used as input & outputs for model building. The model is applied each time a breath profile is collected, and this is used to make a prediction, which is available to the consumer. These predictions are continuously improved because the cycle actively feeds new data into the system, allowing the models to be refined.

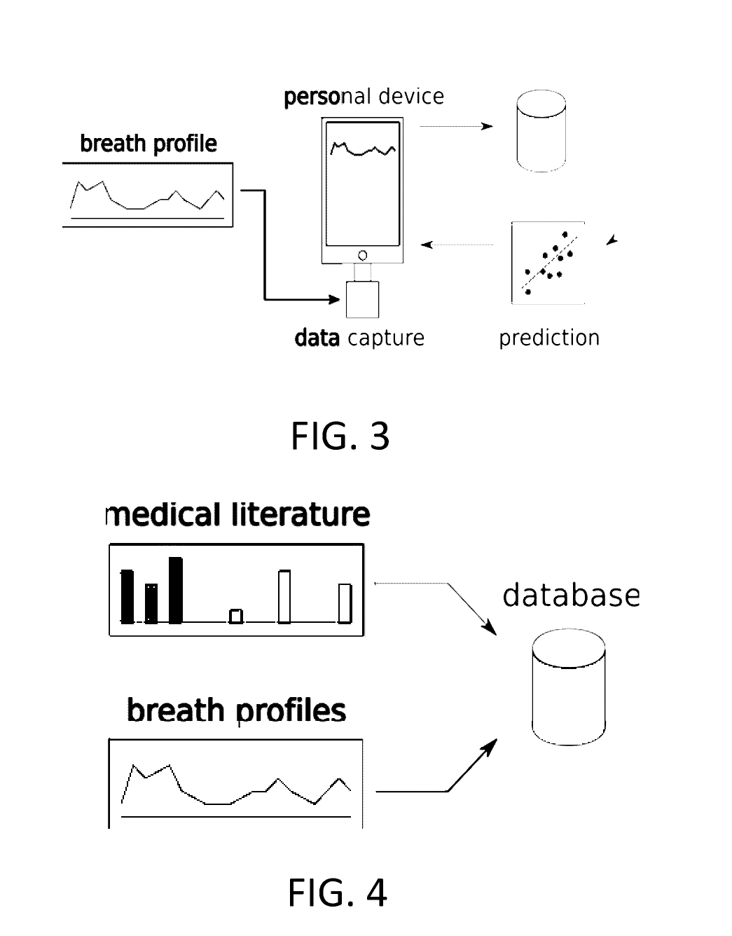

FIG. 3 shows exemplary process for capturing breath profile. Capturing of a breath profile (an input) can be done by various means (e.g. nasal stent or special gum, pacifier, or other device, followed by GC/MS analysis or other interpretative technologies) for obtaining volatile/semi volatile compound spectra from samples. The consumer is assigned a unique and anonymous identifier by means of their phone or other common personal device. The breath profile is loaded onto the device, then transmitted anonymously to the central server. The breath profile is used to make a prediction, which is immediately made available to the consumer. Meanwhile the profile information is stored and associated with the anonymous consumer ID. If it is subsequently followed up by a formal medical diagnosis, the data becomes eligible for inclusion in the model, therefore improving all subsequent predictions.

FIG. 4 shows an exemplary database for use in a PDBDS. The system supports a number of disease indicators (inputs), focusing initially on those with a precedent for correlating volatile molecules with disease. Much of this information is available in the form of publications in the scientific literature or data from clinical trials. This data is typically of high quality, but not always complete or abundant. It will be imported to our database and used to bootstrap the initial model building process, in order to provide prediction value while the data acquisition process is initiated. This process will be repeated with each new disease indication that is added to the system.

### DETAILED DESCRIPTION

The present disclosure generally related to predictive disease breath database systems (PDBDSs) and methods. A PDBDS may accumulate information about the volatile, semi-volatile, and non-volatile organic compounds in breath/saliva. A goal may be to identify disease indications as early as possible, using non-invasive data collection via breath and alert consumers directly for follow-up with a health professional. The database system that can make use of an ever growing collection of empirical evidence to make increasingly accurate predictions, at ever earlier stages, for a growing number of diseases that can be correlated with volatile, semi-volatile, and non-volatile emissions. This may be accomplished using a highly streamlined data collection process using novel devices, combined with a data collection process that is easy and inexpensive for patients and doctors, and cutting edge techniques in machine learning based on contemporary approaches for solving big data problems. Exemplary techniques for extracting volatile and non-volatile chemicals from patients are described in U.S. Pat. No. 9,480,461, entitled “Methods for Extracting Chemicals from Nasal Cavities and Breath” and issued on Nov. 1, 2016, which is incorporated herein by reference.

A readout that is collected from breath samples of consumers may be a spectrum of compounds and/or concentrations that are derived from (gc and lc) mass spectroscopy database and/or olfactory data integration for compound identification, cross referenced to a growing curated list of known compounds. These readouts can be taken at multiple times for any consumer over the course of years, and for multiple consumers. This represents one type of input data to the system. For each of these consumers, the PDBDS may accumulate or “predict” diagnosis events that correspond to diseases/biomarkers of that consumer and also apply learning collectively from other consumers to generate triggered indications as the system continues to learn, and these constitute output data (FIG. 1).

The successful assembly of this database of inputs and outputs makes up the prerequisites for a machine learning campaign. Machine learning techniques may be used to identify profile patterns of compounds that are indicative of early indicators of a future disease. The PDBDS may make use of contemporary deep neural networks, with training/testing set partitioning to verify predictive ability. By including multiple timestamped measurements across the patient database, the PDBDS may be able to determine the maximum extent of our detection capability, i.e. how far back in time we are able to reach with acceptable predictivity.

An important characteristic of machine learning techniques such as deep neural networks is that they are able to identify patterns that are not only counterintuitive, but could not be determined without having access to a large amount of computing power and recent advances in deep learning algorithms. While some relatively straightforward patterns could be determined by expert technicians, the potential level of sensitivity that becomes possible with a large amount of high quality data and computing power represents a difference in kind compared to what is possible with analog data processing methods.

The ability to find counterintuitive patterns for correlating compound spectra with disease indicators can also be extended by augmenting the input conditions with other patient metadata (e.g. simple observables such as age, gender, smoking, diet, or even genetic markers). Clustering based on these additional conditions may improve the ability to subtarget pattern-to-disease correlation. The use of machine learning algorithms allows the possibility of establishing correlations that are counterintuitive and multidimensional, and are not plausible by traditional methods.

One important innovation is the continuous data acquisition process (FIGS. 2 & 3). Our methods for gathering breath sample data from consumers, combined with our ability to link diagnosis events with doctors (another input), allow us to accumulate an ever growing set of data that can be split into training/testing sets for model building, on a near realtime basis. The direct connection that we have with the data gathering process addresses many of the applicability and reproducibility concerns that negatively affect other biomedical modeling exercises. We may rebuild our models regularly as new data becomes available. One process involves iteratively improving our models with increases in data quantity, which forms a virtuous cycle: improved prediction means more successful early diagnoses, which further increases the data quality.

Another input may be aroma (olfactory) and the compound(s) that create the aroma that are aligned with different disease signatures. Using aroma allows for earlier recognition of disease due to aroma often being perceivable prior to compound detection utilizing existing technologies. Inputs can come from the same sources such as research, consumer reporting directly or through social media platforms and others.

In some embodiments, the system is designed to handle multiple disease indications, each of which has its own category of models for making predictions (and can also be used as input metadata, to help subcategorize). As new diseases are added, the system may be pre-populated with data from available sources, such as the medical literature and clinical trials (FIG. 4). Transforming this data into the same form as is used for our own field collection method requires curation, but is highly valuable during the early stages of adding new disease content, especially if the available information about diagnoses is sparse.

One of the benefits of having a continuously learning system that improves the quality of disease models (as well as adding new disease models) is that it becomes possible to re-analyze historical consumer data. When consumers are found to be at risk for an improved or new disease indication, based on previously acquired data, the system will trigger an alert. The consumer will be contacted directly, with a suggestion that they seek medical diagnosis. Use of personal devices (such as phones) gives us a pathway to deliver these notifications.

All of the dimensions of the system are designed to grow over time: as well as the number of disease indications and the volume of patient data, the list of volatile marker compounds may also grow as more relevant chemical structures are discovered. These may be integrated into the profiles, and tagged retroactively from the GC/MS data that corresponds to each of the breath profile datasets.

Gathering the data and storing it in compliance with all regulations regarding anonymity of medical information is a significant challenge: mapping of consumer identifiers with the breath data they generate, and the diagnoses that their doctors make, is a valuable part of the competitive advantage.

Finally, this system may include a financial tracking system that allows for subscriptions payments for participation from users of the system, and it also may allow for integration direct back to users, if desired by system owner, to distribute a financial revenue share, based upon new learning and discoveries that traditionally had only been available to venture capitalists, investors, pharmaceutical companies and other like individuals/companies.

1. A method for detecting disease, comprising:
extracting chemicals from breaths of a plurality of patients over time;
associating one or more of the plurality of patients with a disease;
analyzing the extracted chemicals to identify a predictive marker for the disease based on the assocating.
